(19) United States
(12) Patent Application Publication
(10) Pub. No.: US 2026/0317358 A1
(43) Pub. Date: Oct. 15, 2026

(54) ARESENOPLATIN-1 DERIVITIVES AND METHODS OF TREATMENT THEREWITH

(71) Applicant: Northwestern University, Evanston, IL (US)

(72) Inventors: Denana Miodragovic, Evanston, IL (US); Thomas V. O'Halloran, Evanston, IL (US); Wenan Qiang, Evanston, IL (US)

(21) Appl. No.: 19/646,040

(22) Filed: Apr. 13, 2026

Related U.S. Application Data
(60) Provisional application No. 63/787,582, filed on Apr. 11, 2025.

(51) Int. Cl. C07F 15/00; A61K 31/555; A61P 35/00

(52) U.S. Cl. CPC C07F 15/0093; A61K 31/555; A61P 35/00

(57) ABSTRACT

Provided herein are nitrogenous base derivatives of arsenoplatin-1, and methods of use thereof for the treatment of cancer. In particular, provided herein are pharmaceutical compositions comprising a compound of the structure:

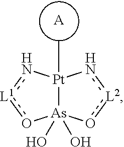

wherein A is a nitrogenous base and L¹ and L² are bridging amidate ligands, and methods of use thereof for the treatment of cancers.

### CROSS-REFERENCE TO RELATED APPLICATION

The present application claims priority to U.S. Provisional Patent Application Ser. No. 63/787,582, filed Apr. 11, 2025, which is herein incorporated by reference in its entirety.

### STATEMENT REGARDING FEDERALLY SPONSORED RESEARCH OR DEVELOPMENT

This invention was made with government support under grant numbers CA202997, CA202995, CA193419 and CA203000 awarded by the National Institutes of Health. The government has certain rights in the invention.

### FIELD

Provided herein are nitrogenous base derivatives of arsenoplatin-1, and methods of use thereof for the treatment of cancer. In particular, provided herein are pharmaceutical compositions comprising a compound of the structure:

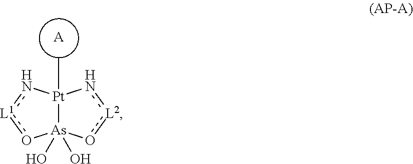

wherein A is a nitrogenous base and L¹ and L² are bridging amidate ligands, and methods of use thereof for the treatment of cancers.

### BACKGROUND

Many cancers require urgent new drugs because they stop responding to drugs currently in clinical practice. The main reason is that cancer cells develop resistance to current medicines through various mechanisms, and drugs with different mechanisms of action are urgently needed. Ovarian cancer is the most lethal gynecological cancer, with a 5-year survival rate of 35%. Ovarian cancer patients initially respond to platinum therapy (Cisplatin, Carboplatin), but in most cases, resistance to drugs will occur.

Arsenoplatin-1 (AP-1) is an anticancer drug that is useful for the treatment of a variety of hematological and solid cancers, including, but not limited to breast cancer, leukemia, colon cancer, ovarian cancer, and renal cancer.

### SUMMARY

Provided herein are nitrogenous base derivatives of arsenoplatin-1, and methods of use thereof for the treatment of cancer. In particular, provided herein are compositions comprising a compound of the structure:

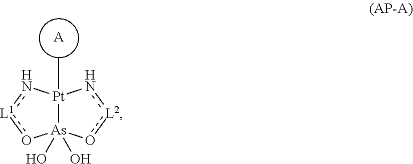

or a tautomer thereof; wherein A is a nitrogenous base and L¹ and L² are bridging amidate ligands. In some embodiments, the tautomer of AP-A is of the structure:

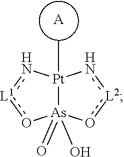

wherein A is a nitrogenous base and L¹ and L² are bridging amidate ligands. In some embodiments, A is a five-membered nitrogen-containing heterocycle. In some embodiments, A is imidazole or an imidazole derivative. In some embodiments, A is selected from 2-methylimidazole, 4-methylimidazole, 5-methylimidazole, 2-ethylimidazole, 1-methylimidazole, 1,2-dimethylimidazole. In some embodiments, A is selected from imidazole, 2-methylimidazole, 4-methylimidazole, 5-methylimidazole, 2-ethylimidazole, 1-methylimidazole, 1,2-dimethylimidazole, histidine, histamine, clotrimazole, ketoconazole, metronidazole, miconazole, pyrazole, 1,2,3-triazole, 1,2,4-triazole, fluconazole, itraconazole, tetrazole, thiazole, oxazole, benzimidazole, albendazole, mebendazole, benzotriazole, 1,2,4-oxadiazole, adenine, guanine, cytosine, thymine, uracil, pyrrole, pyridine, imidazopyridine, and zolpidem. In some embodiments, L¹ and L² are C(O), C—R¹ or C—R², and wherein R¹ and R² are independently selected from hydrogen, halogen, cyano, keto, ester, ether, thiol, thioether, thioester, imino, C₁-C₁₀ alkyl, C₁-C₁₀ alkenyl, alkynyl, alkoxyl, amino, amidyl, immino, sulfonyl, sulfoxyl, phosphoryl, phosphoryl ester, glycosyl, aryl, C₃-C₁₅ cycloalkyl, heteroaryl, and C₃-C₁₅ heterocycloalkyl. In some embodiments, R¹ and R² are each independently selected from a C₁-C₁₀ alkyl. In some embodiments, L¹ and L² are identical functional groups. In some embodiments, L¹ and L² are both —CH3, and the compound is of the structure:

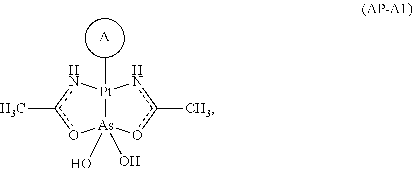

or a tautomer thereof. In some embodiments, the tautomer of AP-A1 is of the structure:

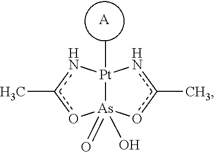

In some embodiments, A is imidazole and the compound is of the structure:

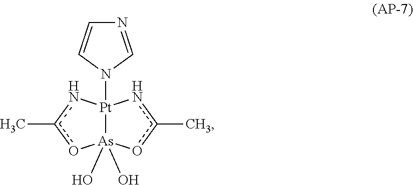

or a tautomer thereof. In some embodiments, the tautomer of AP-7 is of the structure:

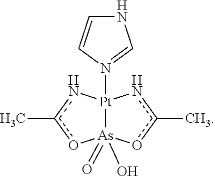

In some embodiments, A is 2-methylimidazole and the compound is of the structure:

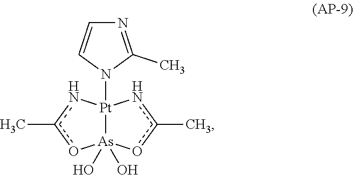

or a tautomer thereof. In some embodiments, the tautomer of AP-9 is of the structure:

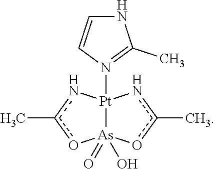

In some embodiments, A is 2-ethylimidazole and the compound is of the structure:

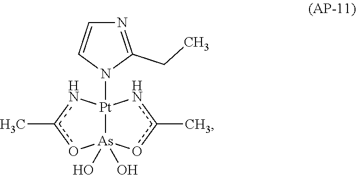

or a tautomer thereof. In some embodiments, the tautomer of AP-11 is of the structure:

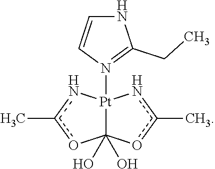

In some embodiments, provided herein are pharmaceutical compositions comprising the composition described herein (e.g., comprising a compound of AP-A, AP-A1, AP-7, AP-9, AP-11, etc.), and a pharmaceutically acceptable carrier. In some embodiments, the pharmaceutical composition is formulated for parenteral administration. In some embodiments, the pharmaceutical composition is formulated for intravenous administration. In some embodiments, the compound is present at a concentration of from about 0.01 μg/mL to about 500 mg/mL (e.g., 0.01 μg/mL, 0.02 μg/mL, 0.05 μg/mL, 0.1 μg/mL, 0.2 μg/mL, 0.5 μg/mL, 1 μg/mL, 2 μg/mL, 5 μg/mL, 10 μg/mL, 20 μg/mL, 50 μg/mL, 100 μg/mL, 200 μg/mL, 500 mg/mL, or values or ranges therebetween).

In some embodiments, provided herein are methods of treating cancer in a subject in need thereof, comprising administering to the subject a therapeutically effective amount of a compound, composition, or pharmaceutical composition described herein (e.g., comprising AP-A, AP-A1, AP-7, AP-9, AP-11, etc.). In some embodiments, the cancer is a solid tumor cancer. In some embodiments, the cancer is selected from the group consisting of ovarian cancer, breast cancer, colon cancer, renal cancer, non-small cell lung cancer, and central nervous system cancer. In some embodiments, the cancer is ovarian cancer. In some embodiments, the cancer is a hematologic malignancy. In some embodiments, the cancer is resistant to one or more platinum-based chemotherapeutic agents. In some embodiments, the one or more platinum-based chemotherapeutic agents comprises cisplatin. In some embodiments, the pharmaceutical composition is co-administered with one or more additional therapies. In some embodiments, the one or more additional therapies are selected from chemotherapeutic agents, immunotherapeutic agents, radiation therapy, and surgical resection.

In some embodiments, provided herein are methods of making/synthesizing a compound or composition described herein (e.g., comprising AP-A, AP-A1, AP-7, AP-9, AP-11, etc.), the method comprising reacting Arsenoplatin-1 (AP-1) with a nitrogenous base A in a solvent system to form the compound. In some embodiments, the solvent system comprises a mixture of methanol and water. In some embodiments, the AP-1 and the nitrogenous base A are reacted in a molar ratio of about 1:1. In some embodiments, the reaction is conducted at a pH of about 7.0 to about 8.0.

### BRIEF DESCRIPTION OF THE DRAWINGS

FIG. 1. Crystal structure of Arsenoplatin-7.

FIG. 2A-E. In vitro testing of AP-7 in the NCI-60 screen.

FIG. 3. IC50 value for AP-7 in A2780 WT and A2780 CDDP cell lines.

FIG. 4. The antitumor activity of AP-7 in an ovarian cancer PDX model. At the end of AP-7 treatment, the tumor weight changes were measured for statistical analysis. **P<0.01.

FIG. 5. Maximum Tolerate Dose (MTD) study for AP-7.

FIG. 6. Exemplary nitrogenous bases suitable for use as the moiety A in the compounds described herein.

FIG. 7. Structure of Arsenoplatin-7 (AP-7), depicting a tautomeric form in which one arsenic-bound oxygen is protonated (As—OH) and the other is deprotonated (As═O), and in which the imidazole ring nitrogen bears a hydrogen atom.

FIG. 8. Crystal structure of Arsenoplatin-9 (AP-9), the 2-methylimidazole derivative.

FIG. 9. Crystal structure of Arsenoplatin-11 (AP-11), the 2-ethylimidazole derivative.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “about” when used in reference to a numerical value means the stated value±10% of the stated value, unless otherwise specified or apparent from the context. For example, “about 7.5” encompasses values from 6.75 to 8.25.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “Arsenoplatin-1” or “AP-1” refers to a platinum (II)-arsenic dinuclear complex having the formula [Pt(μ-NHC(CH₃)O)₂ClAs(OH)₂], in which a platinum (II) center and an arsenic center are bridged by two acetoamidate ligands, and in which a chloride ligand is coordinated to the platinum (II) center. AP-1 and related arsenoplatin compounds have been described in U.S. Pat. No. 10,118,938, which is incorporated by reference in its entirety.

As used herein, the term “Arsenoplatin-A” or “AP-A” refers to the genus of nitrogenous base derivatives of Arsenoplatin-1, in which the chloride ligand of AP-1 is replaced by a nitrogenous base A coordinated to the platinum (II) center through a ring nitrogen atom of the nitrogenous base.

As used herein, the term “Arsenoplatin-7” or “AP-7” refers to the compound of the AP-A genus in which A is imidazole and L¹ and L² are both —CH₃ (i.e., the bridging amidate ligands are acetoamidates), having the molecular formula C₇H₁₅AsN₄O₅Pt and a molecular weight of approximately 505.24 g/mol. AP-7 can exist in tautomeric forms that differ in the position of hydrogen atoms on the imidazole ring nitrogen and/or the arsenic-bound oxygen atoms (e.g., deprotonation/protonation of nitrogen on the imidazole ring; —OH, —O⁻, or ═O bound to As; etc.).

As used herein, the term “Arsenoplatin-9” or “AP-9” refers to the compound of the AP-A genus in which A is 2-methylimidazole and L¹ and L² are both —CH₃. AP-9 can exist in tautomeric forms that differ in the position of hydrogen atoms on the 2-methylimidazole ring nitrogen and/or the arsenic-bound oxygen atoms. AP-9 can exist in tautomeric forms that differ in the position of hydrogen atoms on the 2-methylimidazole ring nitrogen and/or the arsenic-bound oxygen atoms (e.g., deprotonation/protonation of nitrogen on the 2-methylimidazole ring; —OH, —O⁻, or ═O bound to As; etc.).

As used herein, the term “Arsenoplatin-11” or “AP-11” refers to the compound of the AP-A genus in which A is 2-ethylimidazole and L¹ and L² are both —CH₃. AP-11 can exist in tautomeric forms that differ in the position of hydrogen atoms on the 2-ethylimidazole ring nitrogen and/or the arsenic-bound oxygen atoms. AP-11 can exist in tautomeric forms that differ in the position of hydrogen atoms on the 2-ethylimidazole ring nitrogen and/or the arsenic-bound oxygen atoms (e.g., deprotonation/protonation of nitrogen on the 2-ethylimidazole ring; —OH, —O⁻, or ═O bound to As; etc.).

As used herein, the term “bridging amidate ligand” refers to a bidentate ligand that bridges two metal or metalloid centers (e.g., a platinum center and an arsenic center) through coordination at a nitrogen atom to one center and at an oxygen atom to the other center, and that comprises a carbonyl group C(O) bonded to a substituent R. In the context of the AP-A compounds described herein, L¹ and L² are bridging amidate ligands that bridge the platinum (II) center and the arsenic center.

As used herein, the term “acetoamidate” refers to a bridging amidate ligand in which the substituent R on the carbonyl group is a methyl group (—CH₃). In the compounds AP-7, AP-9, and AP-11, L¹ and L² are each acetoamidate ligands.

As used herein, the term “tautomer” refers to constitutional isomers of a compound that differ only in the position of one or more hydrogen atoms and the associated redistribution of bonding electrons. In the context of the arsenoplatin compounds described herein, tautomeric forms may arise from (i) prototropic tautomerism of the nitrogenous base A (e.g., migration of a hydrogen atom between ring nitrogen atoms of an imidazole), and/or (ii) prototropic tautomerism at the arsenic center, wherein the arsenic-bound oxygen atoms may exist in protonated (As—OH) or deprotonated (As═O) forms. Without being bound by theory, crystal structure analyses of AP-7, AP-9, and AP-11 indicate that differences in As—O bond lengths are consistent with the existence of tautomeric forms in which one arsenic-bound oxygen is protonated (As—OH) and the other is deprotonated (As═O). Reference herein to a compound “or a tautomer thereof” encompasses all such tautomeric forms, whether they exist in solution, in the solid state, or in equilibrium.

As used herein, the term “five-membered nitrogen-containing heterocycle” or “five-membered nitrogen-containing heterocyclic compound” refers to any aromatic or non-aromatic cyclic compound comprising a five-membered ring containing at least one nitrogen atom as a ring member. Examples include, but are not limited to, imidazole, pyrazole, 1,2,3-triazole, 1,2,4-triazole, tetrazole, thiazole, oxazole, isoxazole, isothiazole, pyrrole, and substituted or fused derivatives thereof.

As used herein, the term “nitrogenous base” refers to any organic compound containing one or more nitrogen atoms within a heterocyclic ring structure, capable of participating in hydrogen bonding or proton exchange. This includes, but is not limited to, purines, pyrimidines, imidazoles, pyrazoles, triazoles, tetrazoles, oxazoles, thiazoles, and their substituted or fused derivatives. Such bases may be naturally occurring, synthetic, or chemically modified, and are characterized by their basicity, nucleophilicity, or ability to coordinate with metal ions or participate in biological interactions.

As used herein, the term “imidazole derivative” refers to any compound structurally based on the imidazole ring, a five-membered aromatic heterocycle containing two non-adjacent nitrogen atoms at the 1 and 3 positions. Imidazole derivatives include compounds in which one or more hydrogen atoms on the imidazole ring are substituted with alkyl, aryl, heteroaryl, halogen, hydroxyl, nitro, amino, or other functional groups, as well as compounds in which the imidazole ring is fused with additional aromatic or non-aromatic rings. These derivatives may exhibit biological, chemical, or pharmacological activity and can serve various functions, including as ligands, intermediates, or active pharmaceutical ingredients.

As used herein, the term “pharmaceutically acceptable carrier” refers to non-toxic solid, semisolid, or liquid filler, diluent, encapsulating material, formulation auxiliary, excipient, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. A “pharmaceutical composition” typically comprises at least one active agent (e.g., the copolymers described herein) and a pharmaceutically acceptable carrier.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the term “parenteral administration” refers to administration by any route other than through the alimentary canal, including, but not limited to, intravenous, intramuscular, subcutaneous, intratumoral, intraperitoneal, intradermal, and intrathecal administration.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “platinum-based chemotherapeutic agent” refers to any chemotherapeutic compound comprising a platinum metal center as part of its active pharmacophore. Examples of platinum-based chemotherapeutic agents include, but are not limited to, cisplatin (cis-diamminedichloroplatinum (II)), carboplatin, oxaliplatin, nedaplatin, lobaplatin, and satraplatin.

As used herein, a cancer is “resistant” to a chemotherapeutic agent when the cancer cells exhibit reduced sensitivity to the agent relative to a reference cancer cell population, as measured by any art-recognized assay, including, but not limited to, IC₅₀ assays, cell viability assays, or clinical response criteria. Resistance may be intrinsic or acquired.

As used herein, the term “solid tumor cancer” refers to a neoplasm that forms a discrete mass of tissue and does not include liquid or circulating cells at the time of diagnosis. Examples of solid tumor cancers include, but are not limited to, ovarian cancer, breast cancer, colon cancer, renal cancer, lung cancer, central nervous system cancers, pancreatic cancer, liver cancer, head and neck cancers, and prostate cancer.

As used herein, the term “hematologic malignancy” refers to a cancer that originates in blood-forming tissues or immune cells, including, but not limited to, leukemias, lymphomas, and multiple myeloma.

### DETAILED DESCRIPTION

Provided herein are nitrogenous base derivatives of arsenoplatin-1, and methods of use thereof for the treatment of cancer. In particular, provided herein are pharmaceutical compositions comprising a compound of the structure:

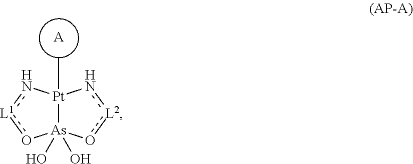

wherein A is a nitrogenous base and L¹ and L² are bridging amidate ligands, and methods of use thereof for the treatment of cancers.

Arsenoplatin-A (AP-A) is a genus of nitrogenous base derivatives of Arsenoplatin-1. A nitrogenous base is attached to the platinum (II) center of the platinum center. In some embodiments, provided herein is a compound of the structure:

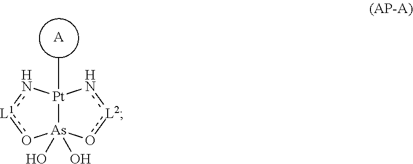

wherein A is a nitrogenous base and L¹ and L² are bridging amidate ligands.

In some embodiments, the compound of the structure AP-A, or a tautomer thereof, is provided. Without being bound by theory, the compounds described herein may exist as tautomers that differ in the position of hydrogen atoms on the nitrogenous base ring and/or on the arsenic-bound oxygen atoms. For example, in certain tautomeric forms, one arsenic-bound oxygen may be protonated (As—OH) while the other is deprotonated (As═O), and the position of hydrogen atoms on the ring nitrogen atoms of the nitrogenous base may differ between tautomers. All tautomeric forms of the compounds described herein are encompassed by the present disclosure. For example, in some embodiments, AP-A is of the tautomeric form:

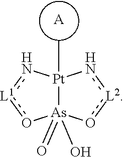

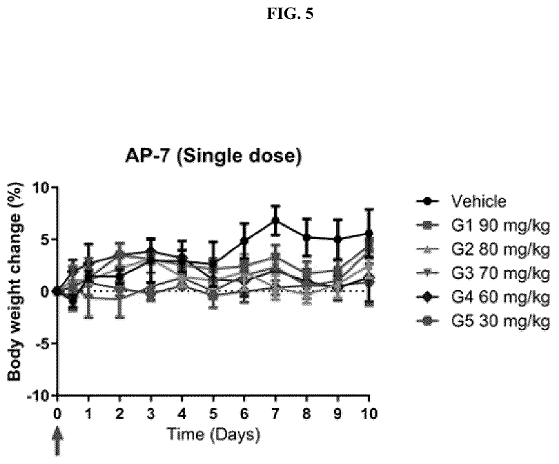

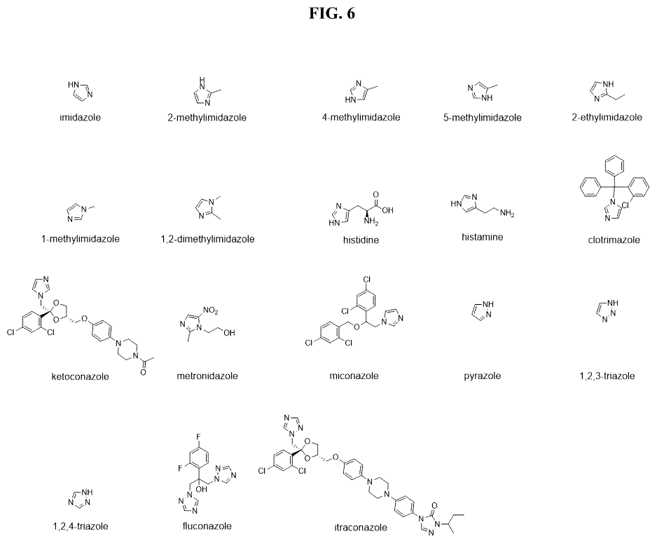

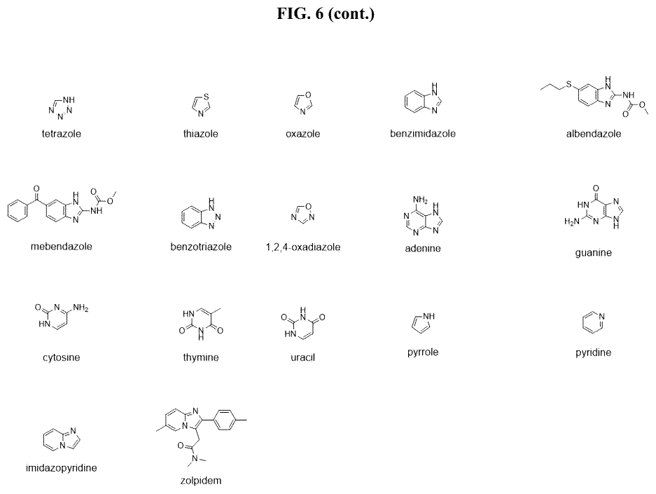

In some embodiments, A is imidazole or an imidazole derivative. In some embodiments, the imidazole derivative is selected from imidazole, 2-methylimidazole, 4-methylimidazole, 5-methylimidazole, 2-ethylimidazole, 1-methylimidazole, 1,2-dimethylimidazole, histidine, histamine, clotrimazole, ketoconazole, metronidazole, miconazole. In some embodiments, A is a nitrogenous base selected from imidazole, 2-methylimidazole, 4-methylimidazole, 5-methylimidazole, 2-ethylimidazole, 1-methylimidazole, 1,2-dimethylimidazole, histidine, histamine, clotrimazole, ketoconazole, metronidazole, miconazole, pyrazole, 1,2,3-triazole, 1,2,4-triazole, fluconazole, itraconazole, tetrazole, thiazole, oxazole, benzimidazole, albendazole, mebendazole, benzotriazole, 1,2,4-oxadiazole, adenine, guanine, cytosine, thymine, uracil, pyrrole, pyridine, imidazopyridine, and zolpidem. Exemplary nitrogenous bases are depicted in FIG. 6. In some embodiments, the A moiety is bonded to the Pt center at a ring N of the nitrogenous base; adjustment of the charge and structure of the A moieties upon bonding to the Pt center at a ring N of the nitrogenous base will be understood in the field.

In some embodiments, A is a five-membered nitrogen-containing heterocyclic compound. In some embodiments, the five-membered nitrogen-containing heterocyclic compound is selected from imidazole, pyrazole, 1,2,3-triazole, 1,2,4-triazole, tetrazole, thiazole, oxazole, and substituted derivatives thereof.

In some embodiments, L¹ and L² are C(O), C—R¹ or C—R², and wherein R¹ and R² are selected from hydrogen, halogen, cyano, keto, ester, ether, thiol, thioether, thioester, imino, C₁-C₁₀ alkyl, C₁-C₁₀ alkenyl, alkynyl, alkoxyl, amino, amidyl, immino, sulfonyl, sulfoxyl, phosphoryl, phosphoryl ester, glycosyl, aryl, C₃-C₁₅ cycloalkyl, heteroaryl, and C₃-C₁₅ heterocycloalkyl. In some embodiments, L¹ and L² (and R¹ and R²) are identical functional groups.

In some embodiments, R¹ and R² are each independently selected from a C₁-C₁₀ alkyl (e.g., C₁, C₂, C₃, C₄, C₅, C₆, C₇, C₈, C₉, C₁₀, or ranges therebetween).

In some embodiments, L¹ and L² are both —CH₃, and provided herein is a compound of the structure:

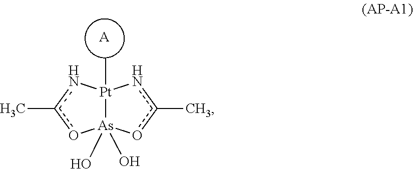

wherein A is a nitrogenous base. In some embodiments, AP-A1 exists as a tautomer in which a hydrogen atom is present on a ring nitrogen of the imidazole and/or one arsenic-bound oxygen is deprotonated (As═O). An exemplary tautomer of AP-A1 has the structure:

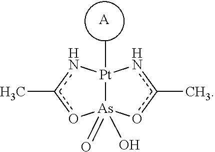

Other tautomers of AP-A1 are within the scope herein.

Arsenoplatin-7 is an exemplary imidazole derivative of Arsenoplatin-1. It is structurally different from all platinum drugs because, besides the aqueous and active form of anticancer drug arsenic trioxide attached to the platinum center, it also has the imidazole ring attached to the platinum (II). In some embodiments, provided herein is a compound of the structure:

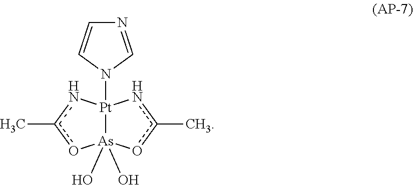

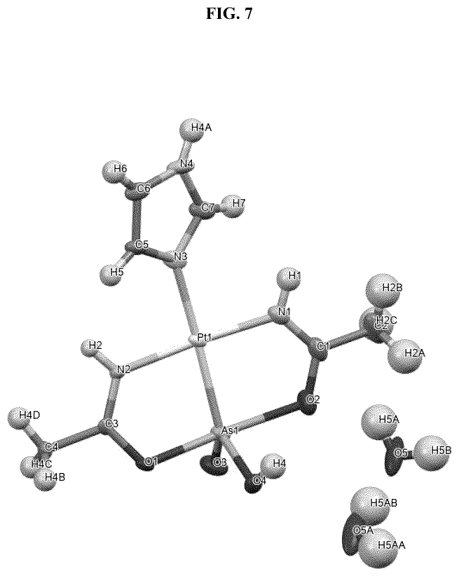

AP-7 has the molecular formula C₇H₁₅AsN₄O₅Pt and a molecular weight of approximately 505.24 g/mol. In some embodiments, AP-7 exists as a tautomer in which a hydrogen atom is present on a ring nitrogen of the imidazole and/or one arsenic-bound oxygen is deprotonated (As═O), as depicted, for example, in FIG. 7. An exemplary tautomer of AP-7 has the structure:

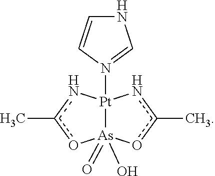

Other tautomers of AP-7 are within the scope herein.

In some embodiments, provided herein is a compound that is Arsenoplatin-9 (AP-9), in which A is 2-methylimidazole and L¹ and L² are both —CH₃. AP-9 has the structure:

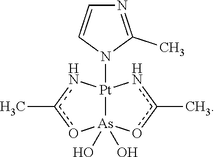

In some embodiments, AP-9 exists as a tautomer in which a hydrogen atom is present on a ring nitrogen of the imidazole and/or one arsenic-bound oxygen is deprotonated (As═O), as depicted An exemplary tautomer of AP-9 within the scope herein has the structure:

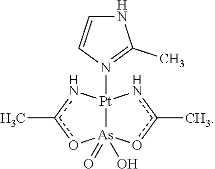

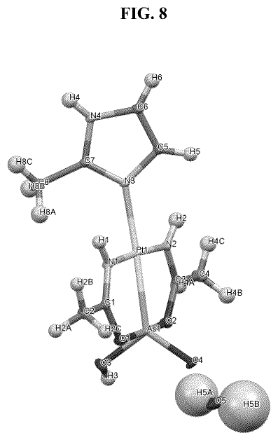

Other tautomers of AP-9 are within the scope herein. The crystal structure of AP-9 is depicted in FIG. 8.

In some embodiments, provided herein is a compound that is Arsenoplatin-11 (AP-11), in which A is 2-ethylimidazole and L¹ and L² are both —CH₃. AP-11 has the structure:

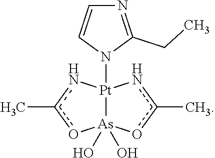

In some embodiments, AP-11 exists as a tautomer in which a hydrogen atom is present on a ring nitrogen of the imidazole and/or one arsenic-bound oxygen is deprotonated (As═O), as depicted An exemplary tautomer of AP-11 within the scope herein has the structure:

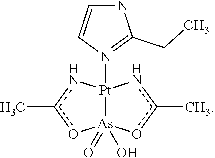

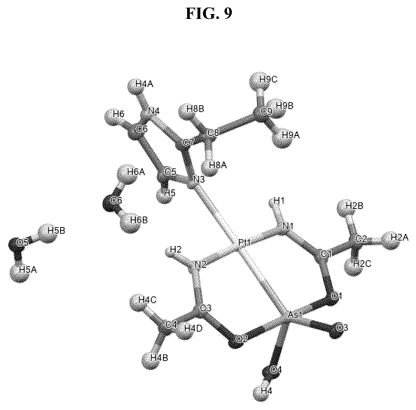

Other tautomers of AP-11 are within the scope herein. The crystal structure of AP-9 is depicted in FIG. 9.

Without being bound by theory, crystal structure analyses of AP-9 and AP-11, which share the same structural pattern as AP-7, indicate that differences in bond lengths for As—O in the structures are consistent with one bond being a single bond (As—OH) and the other being a double bond (As═O). Accordingly, the tautomeric forms depicted herein for AP-7, AP-9, and AP-11 reflect the structural assignments supported by single-crystal X-ray diffraction data for all three compounds.

In some embodiments, AP-9 and AP-11 are synthesized by the same general procedure as AP-7, comprising the reaction of Arsenoplatin-1 (AP-1) with the respective nitrogenous base (2-methylimidazole or 2-ethylimidazole) in a methanol-water solvent mixture at a similar pH range.

In some embodiments, pharmaceutical compositions comprising AP-7 and a pharmaceutical carrier.

Cis-diamminedichloroplatinum (II) (“Cisplatin”) and arsenic trioxide (As₂O₃) are highly successful agents for treatment of cancer. Cisplatin is used in combination chemotherapy to treat ovarian, testicular, head, neck, and bladder cancers. Unfortunately, these and other cancers frequently develop resistance to this widely used agent and there are intensive efforts to develop new agents that overcome this resistance. Arsenic trioxide, which was discovered as a traditional Chinese medicine, is a front-line treatment for acute promyelocytic leukemia and has also shown preliminary efficacy in the treatment of blood cancers such as multiple myeloma and myelodysplastic syndromes.

Both compounds induce apoptotic cell death, but through different pathways. Cisplatin reacts with DNA and causes intra- and inter-strand DNA cross-links. The principal component of aqueous solutions of As2O3 at pH 7, arsenous acid, at low concentrations reacts with and triggers degradation of key zinc-dependent regulatory proteins and inhibits angiogenesis, migration and invasion. At higher concentrations, arsenous acid triggers apoptosis through pathways that involve elevated levels of reactive oxygen species in mitochondria.

Arsenoplatin anti-cancer agents have been described in, for example, U.S. Pat. Nos. 9,499,957, 10,118,938, and 9,725,475 (each of which is incorporated by reference in their entireties). The nitrogenous base derivatives of AP-1 described herein (e.g., AP-A, AP-A1, AP-7, AP-9, AP-11, tautomers of the aforementioned, etc.) are structurally and functionally distinct from existing arsenoplatin compounds because besides the aqueous and active form of anticancer drug arsenic trioxide attached to the platinum center, they also have the nitrogenous base (e.g., imidazole ring, imidazole derivative, etc.) attached to the platinum (II). Imidazole is an aromatic diazole and alkaloid with anticancer properties. Imidazole-based drugs have shown potential in treating cancer and are being developed as safer alternatives to chemotherapy. Imidazoles can stop cell growth and division by: Binding to protein molecules, Interfering with DNA synthesis, and Pi-pi stacking. However, The nitrogenous base derivatives of AP-1 described herein (e.g., AP-A, AP-A1, AP-7, AP-9, AP-11, tautomers of the aforementioned, etc.) are also structurally and functionally distinct from imidazole-based drugs such as dacarbazine, fadrozole, and ketoconazole. Experiments conducted during development of embodiments herein demonstrate that the exemplary compound AP-7 is potent against a wide range of cancer types and is well tolerated by mammalian subjects.

In some embodiments, provided herein are pharmaceutical compositions of the nitrogenous base derivatives of AP-1 described herein (e.g., AP-A, AP-A1, AP-7, AP-9, AP-11, tautomers of the aforementioned, etc.) for administration to mammals to treat cancer and related conditions. In a preferred embodiment, a composition for administration is a pharmaceutical composition, preferably in a single unit dosage form. Pharmaceutical compositions and single unit dosage forms can comprise a prophylactically or therapeutically effective amount of the nitrogenous base derivatives of AP-1 described herein (e.g., AP-A, AP-A1, AP-7, AP-9, AP-11, tautomers of the aforementioned, etc.), and a typically one or more pharmaceutically acceptable carriers or excipients or diluents.

A pharmaceutical composition comprising a nitrogenous base derivative of AP-1 described herein (e.g., AP-A, AP-A1, AP-7, AP-9, AP-11, tautomers of the aforementioned, etc.) can be administered by any route according to the judgment of those of skill in the art, including but not limited to orally, intravenously, intragastrically, intraduodenally, intraperitoneally or intracerebroventricularly.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, PBS, and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

The disclosure further encompasses administration of pharmaceutical compositions of the nitrogenous base derivatives of AP-1 described herein (e.g., AP-A, AP-A1, AP-7, AP-9, AP-11, tautomers of the aforementioned, etc.) in single unit dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions comprising a nitrogenous base derivative of AP-1 described herein (e.g., AP-A, AP-A1, AP-7, AP-9, AP-11, tautomers of the aforementioned, etc.) and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, PBS, etc. Such compositions and dosage forms will contain a prophylactically or therapeutically effective amount of a preparation of the described compounds having anti-cancer effect, together with a suitable amount of carrier so as to provide the form for proper administration to the patient.

The formulation should suit the mode of administration. In a preferred embodiment, the pharmaceutical compositions comprising a nitrogenous base derivative of AP-1 described herein (e.g., AP-A, AP-A1, AP-7, AP-9, AP-11, tautomers of the aforementioned, etc.) are sterile and prepared in a form suitable for administration to a subject, preferably an animal subject, more preferably a mammalian subject, and most preferably a human subject. Besides humans, preferred animal subjects include horses, birds, cats, dogs, rats, hamsters, mice, guinea pigs, cows, and pigs.

A pharmaceutical composition comprising a nitrogenous base derivative of AP-1 described herein (e.g., AP-A, AP-A1, AP-7, AP-9, AP-11, tautomers of the aforementioned, etc.) are formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, a composition comprising AP-7 is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for oral administration to human beings. Typically, compositions for oral administration are solid dosage forms or solutions in sterile isotonic aqueous buffer.

Examples of dosage forms include, but are not limited to: tablets; caplets; capsules, such as soft elastic gelatin capsules or hard capsules; dropping pills; cachets; troches; lozenges; dispersions; suppositories; ointments; cataplasms (poultices); pastes; powders; dressings; creams; plasters; solutions; patches; aerosols (for example, nasal sprays or inhalers); gels; liquid dosage forms suitable for oral or mucosal administration to a patient, including suspensions (for example, aqueous or non-aqueous liquid suspensions, oil-in-water emulsions, or a water-in-oil liquid emulsions), solutions, and elixirs; liquid dosage forms suitable for parenteral or intravenous administration to a patient; and sterile solids (for example, crystalline or amorphous solids or granular forms) that can be reconstituted to provide liquid dosage forms suitable for parenteral or intravenous administration to a patient.

The composition, shape, and type of dosage forms of a preparation of a nitrogenous base derivative of AP-1 described herein (e.g., AP-A, AP-A1, AP-7, AP-9, AP-11, tautomers of the aforementioned, etc.) will typically vary depending on their use. For example, a dosage form used in the acute treatment of a cancer or related disorder may contain larger amounts of one or more of a preparation of the disclosed compounds than a dosage form used in the chronic treatment of the same disease. Also, the therapeutically effective dosage form may vary among different types of diseases or disorders. Similarly, a parenteral dosage form may contain smaller amounts of one or more of the active than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

In some embodiments, the ingredients of compositions comprising a preparation comprising a nitrogenous base derivative of AP-1 described herein (e.g., AP-A, AP-A1, AP-7, AP-9, AP-11, tautomers of the aforementioned, etc.) are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutical compositions comprising a nitrogenous base derivative of AP-1 described herein (e.g., AP-A, AP-A1, AP-7, AP-9, AP-11, tautomers of the aforementioned, etc.) used in methods that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In certain embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms are prepared by combining the active ingredient(s) (that is, a preparation of the disclosed compound(s)) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Examples of excipients that can be used in oral dosage forms include, but are not limited to, binders, fillers, disintegrants, and lubricants. Binders suitable for use in pharmaceutical compositions comprising a nitrogenous base derivative of AP-1 described herein (e.g., AP-A, AP-A1, AP-7, AP-9, AP-11, tautomers of the aforementioned, etc.) and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions comprising a nitrogenous base derivative of AP-1 described herein (e.g., AP-A, AP-A1, AP-7, AP-9, AP-11, tautomers of the aforementioned, etc.) include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

In some embodiments, disintegrants are used in compositions comprising a nitrogenous base derivative of AP-1 described herein (e.g., AP-A, AP-A1, AP-7, AP-9, AP-11, tautomers of the aforementioned, etc.) to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions comprising a nitrogenous base derivative of AP-1 described herein (e.g., AP-A, AP-A1, AP-7, AP-9, AP-11, tautomers of the aforementioned, etc.) include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions comprising a nitrogenous base derivative of AP-1 described herein (e.g., AP-A, AP-A1, AP-7, AP-9, AP-11, tautomers of the aforementioned, etc.) include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The amount of the composition in the methods herein which will be effective in the prevention, treatment, management, or amelioration of a cancer disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems.

Typical dosage forms for administration in methods of the invention comprise a composition of the invention in an amount within the range of from about 0.001 mg to about 500 mg of the disclosed compounds per day, 0.10 mg to 300 mg of the disclosed compounds per day, or 1.0 mg to 200 mg of the disclosed compounds per day given as a single once-a-day dose or as divided doses throughout the day. Particular dosage forms of the invention have incremental variations from about 0.001, 0.01, 0.1, 0.2, 0.25, 0.3, 0.5, 0.75, 1.0, 2.0, 2.5, 3.0, 5.0, 7.5, 10.0, 15.0, 20.0, 25.0, 40.0, 50.0, 60.0, 75.0, 100, 125, 150, 175, 200, 250, 300, 400, and 500 mg of AP-7, as well as incremental dosage variations thereof.

Exemplary dosage forms of the invention having a liquid formulation include 1, 3, 5, 7.5, 10, 15, 20, 50, 75, and 100 ml of a liquid composition of AP-7 having a concentration ranging from about 0.01 μg/ml to about 500 mg/ml. The preferred concentrations of such liquid compositions will depend upon the dissolution characteristics of the medium, which will determine the upper limit of pharmaceutically acceptable concentrations of a nitrogenous base derivative of AP-1 described herein (e.g., AP-A, AP-A1, AP-7, AP-9, AP-11, tautomers of the aforementioned, etc.) in such compositions. Consequently, alternative, pharmaceutically acceptable, concentrations of a nitrogenous base derivative of AP-1 described herein (e.g., AP-A, AP-A1, AP-7, AP-9, AP-11, tautomers of the aforementioned, etc.) in liquid compositions that are lower, as well as higher, than that stated herein are also contemplated by the present invention.

In the case of liquid dosage forms, suitable concentrations of a nitrogenous base derivative of AP-1 described herein (e.g., AP-A, AP-A1, AP-7, AP-9, AP-11, tautomers of the aforementioned, etc.) are suspended or dissolved in pharmaceutically acceptable carrier media, such as water, phosphate buffered saline, saline, and the like. Furthermore, suitable concentrations of a nitrogenous base derivative of AP-1 described herein (e.g., AP-A, AP-A1, AP-7, AP-9, AP-11, tautomers of the aforementioned, etc.) are suspended or dissolved under physiologically and physiochemically appropriate conditions.

To the extent that solubility presents a barrier to any embodiments herein, particle size reduction of a nitrogenous base derivative of AP-1 described herein (e.g., AP-A, AP-A1, AP-7, AP-9, AP-11, tautomers of the aforementioned, etc.) to increase solvent-accessible surface area and solvation potential can improve solubility. Methods, techniques and instrumentation are known in the art for achieving particle size reduction (for example, micronization) can be useful for achieving particle size reduction for the disclosed compounds.

Exemplary doses of a composition of a nitrogenous base derivative of AP-1 described herein (e.g., AP-A, AP-A1, AP-7, AP-9, AP-11, tautomers of the aforementioned, etc.) include microgram or milligram amounts of the disclosed compound(s) per kilogram of subject or sample weight. For a composition used in the invention, the dosage administered to a patient can be administered from about 0.001 mg/kg to about 10 mg/kg. In certain embodiments, the dosage can be from about 0.02 mg/kg to about 4.0 mg/kg of the patient's body weight, based on weight and purity of the nitrogenous base derivative of AP-1 described herein (e.g., AP-A, AP-A1, AP-7, AP-9, AP-11, tautomers of the aforementioned, etc.) preparation in the composition.

The composition can be administered as a single once-a-day dose or as divided doses throughout a day. In some embodiments, the daily dose is administered twice daily in equally divided doses. In other embodiments, the daily dose is administered three times per day. In particular embodiments, the daily dose is administered three times per day in equally divided doses. In particular embodiments, the daily dose is administered four times per day in equally divided doses. The actual dosage can be determined by a practitioner of skill in the art according to, for example, the subjects age, body weight, body mass index, or other factors. In certain embodiments, administration of a composition in the invention may be repeated daily. In certain embodiments and the administrations may be separated by at least 1 day, 2 days or 3 days. An effective amount of a composition described herein will provide therapeutic benefit without causing substantial toxicity. Toxicity of a composition comprising a nitrogenous base derivative of AP-1 described herein (e.g., AP-A, AP-A1, AP-7, AP-9, AP-11, tautomers of the aforementioned, etc.) can be determined by standard pharmaceutical procedures in cell cultures or experimental animals, for example, by determining the LD₅₀ (that is, the dose lethal to 50% of the population) or the LD₁₀₀ (that is, the dose lethal to 100% of the population).

The descriptions of exemplary doses are merely alternative descriptions that may be used optionally at the discretion of the physician and are not intended to conflict or supersede other descriptions of doses disclosed herein.

A composition comprising a nitrogenous base derivative of AP-1 described herein (e.g., AP-A, AP-A1, AP-7, AP-9, AP-11, tautomers of the aforementioned, etc.) can be administered alone or in combination with other treatments, either simultaneously or sequentially dependent upon the condition to be treated.

In some embodiments, a nitrogenous base derivative of AP-1 (e.g., AP-A, AP-A1, AP-7, AP-9, AP-11, tautomers of the aforementioned, etc.) pharmaceutical composition is administered to a subject for the treatment of a cancer. Certain embodiments herein are directed to administration of a nitrogenous base derivative of AP-1 (e.g., AP-A, AP-A1, AP-7, AP-9, AP-11, tautomers of the aforementioned, etc.) pharmaceutical composition to a subject with cancer, in remission from cancer, or at elevated risk of cancer. In some embodiments, a nitrogenous base derivative of AP-1 (e.g., AP-A, AP-A1, AP-7, AP-9, AP-11, tautomers of the aforementioned, etc.) pharmaceutical composition is administered as part of therapeutic or prophylactic regimen for the treatment or prevention of acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myeloproliferative disorders, colon cancer, breast cancer (e.g., triple negative breast cancer, etc.) colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer.

In some embodiments, a nitrogenous base derivative of AP-1 (e.g., AP-A, AP-A1, AP-7, AP-9, AP-11, tautomers of the aforementioned, etc.) pharmaceutical composition is administered to a subject suffering from a cancer that is resistant to one or more platinum-based chemotherapeutic agents. In some embodiments, the cancer is resistant to cisplatin. In some embodiments, the cancer is cisplatin-resistant ovarian cancer.

In some embodiments, a nitrogenous base derivative of AP-1 (e.g., AP-A, AP-A1, AP-7, AP-9, AP-11, tautomers of the aforementioned, etc.) pharmaceutical composition is co-administered along with administration of a chemotherapy agent. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Rucaparib (Rubraca), Niraparib (Zejula), Talazoparib (Talzenna), Veliparib Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Embodiments herein further relate to methods for using a nitrogenous base derivative of AP-1 (e.g., AP-A, AP-A1, AP-7, AP-9, AP-11, tautomers of the aforementioned, etc.) pharmaceutical composition in combination with radiation therapy for inhibiting abnormal cell growth or treating the hyperproliferative disorder in the mammal. Techniques for administering radiation therapy are known in the art, and these techniques can be used in the combination therapy described herein. The administration of a nitrogenous base derivative of AP-1 (e.g., AP-A, AP-A1, AP-7, AP-9, AP-11, tautomers of the aforementioned, etc.) pharmaceutical composition in this combination therapy can be determined as described herein. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

A nitrogenous base derivative of AP-1 (e.g., AP-A, AP-A1, AP-7, AP-9, AP-11, tautomers of the aforementioned, etc.) pharmaceutical composition may also be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with an AP-7 pharmaceutical composition described herein. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-10, MMP-11, MMP-12, and MMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, medicaments which are administered in conjunction with a nitrogenous base derivative of AP-1 (e.g., AP-A, AP-A1, AP-7, AP-9, AP-11, tautomers of the aforementioned, etc.) pharmaceutical composition described herein include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatoires, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl) ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon

Exemplary therapeutic agents useful for a combination therapy with a nitrogenous base derivative of AP-1 (e.g., AP-A, AP-A1, AP-7, AP-9, AP-11, tautomers of the aforementioned, etc.) pharmaceutical composition described herein include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Other suitable therapeutic agents for coadministration with a nitrogenous base derivative of AP-1 (e.g., AP-A, AP-A1, AP-7, AP-9, AP-11, tautomers of the aforementioned, etc.) pharmaceutical composition also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated for co-administration with a nitrogenous base derivative of AP-1 (e.g., AP-A, AP-A1, AP-7, AP-9, AP-11, tautomers of the aforementioned, etc.) pharmaceutical composition include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated for co-administration with a nitrogenous base derivative of AP-1 (e.g., AP-A, AP-A1, AP-7, AP-9, AP-11, tautomers of the aforementioned, etc.) pharmaceutical composition include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-lactam antibiotics, an agent comprising an aminoglycoside, protein synthesis inhibitors, drugs used in the chemotherapy of tuberculosis, Mycobacterium avium complex disease, and leprosy, antifungal agents, antiviral agents including nonretroviral agents and antiretroviral agents.

Examples of therapeutic antibodies that can be combined with a nitrogenous base derivative of AP-1 (e.g., AP-A, AP-A1, AP-7, AP-9, AP-11, tautomers of the aforementioned, etc.) pharmaceutical composition include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

In some embodiments, provided herein are methods of making a compound according to the present disclosure, comprising reacting Arsenoplatin-1 (AP-1) with a nitrogenous base A in a solvent system to form the compound. In some embodiments, the solvent system comprises a mixture of methanol and water. In some embodiments, the methanol and water are present in a volume ratio of about 1:1. In some embodiments, the AP-1 and the nitrogenous base A are reacted in a molar ratio of about 1:1. In some embodiments, the reaction is conducted at a pH of about 7.0 to about 8.0. In some embodiments, the pH is about 7.5. In some embodiments, the reaction is conducted at a temperature of about 40° C. to about 60° C. In some embodiments, the temperature is about 50° C.

In some embodiments, the nitrogenous base A is selected from imidazole, 2-methylimidazole, and 2-ethylimidazole. In some embodiments, the nitrogenous base A is imidazole and the compound produced is Arsenoplatin-7 (AP-7). In some embodiments, the nitrogenous base A is 2-methylimidazole and the compound produced is Arsenoplatin-9 (AP-9). In some embodiments, the nitrogenous base A is 2-ethylimidazole and the compound produced is Arsenoplatin-11 (AP-11). In some embodiments, the compound produced is a AP-A tautomer described herein.

### EXPERIMENTAL

### Example 1

Arsenoplatin-7 was synthesized in the reaction between Arsenoplatin-1 (AP-1) and imidazole in a 1:1 mole ratio in a methanol-water mixture (v/v 1:1). The pH was adjusted to 7.5, and the reaction mixture was left overnight at 50° C. with reflux. The next day, the solution was filtered, transferred to a glass beaker, covered with parafilm with holes, and left in the hood for slow evaporation.

### Example 2

### Single Crystal X-Ray Structure Determination of AP-7

A suitable crystal of C₇H₁₅AsN₄O₅Pt was mounted on a XtaLAB Synergy R, DW system, HyPix diffractometer. The crystal was kept at 99.99 (16) K during data collection. Using Olex2, the structure was solved with the XT structure solution program using Intrinsic Phasing and refined with the SHELXL refinement package using Least Squares minimisation.

Crystal Data for C₇H₁₅AsN₄O₅Pt (M=505.24): monoclinic, space group Cc (no. 9), a=9.7991 (3) Å, b=21.1497 (7) Å, c=7.1450 (3) Å, β=109.735 (4) °, V=1393.81 (9) Å³, Z=4, T=99.99 (16) K, μ(Mo Kα)=12.441 mm-1, Dcalc=2.408 g/mm³, 22727 reflections measured (4.818≤2Θ≤68.418), 4960 unique (Rint=0.0522, Rsigma=0.0532) which were used in all calculations. The final R1 was 0.0388 (I>2σ(I)) and wR2 was 0.0887.

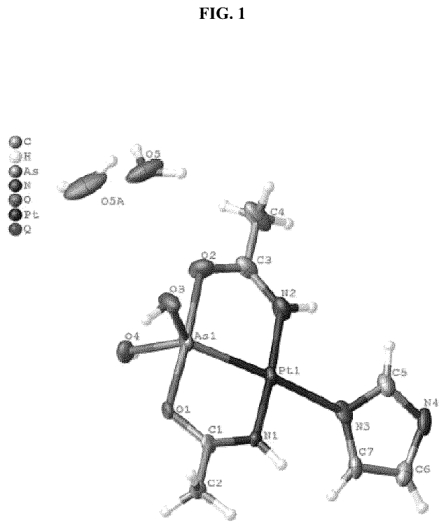

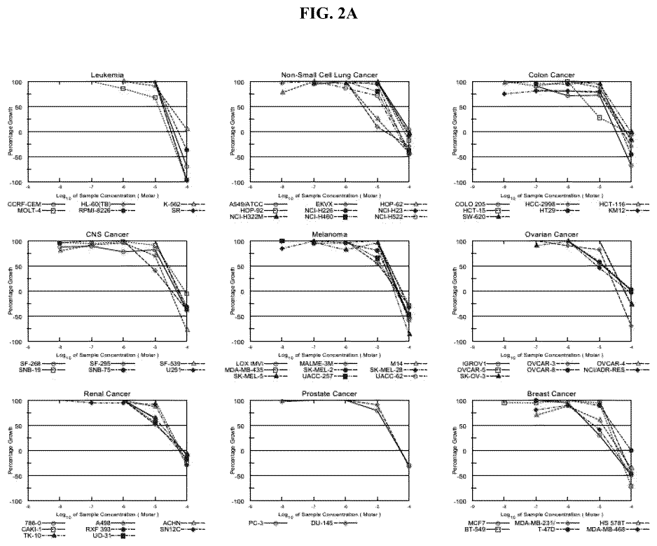

The crystal structure is depicted in FIG. 1.

### Example 3

### Biological Activity

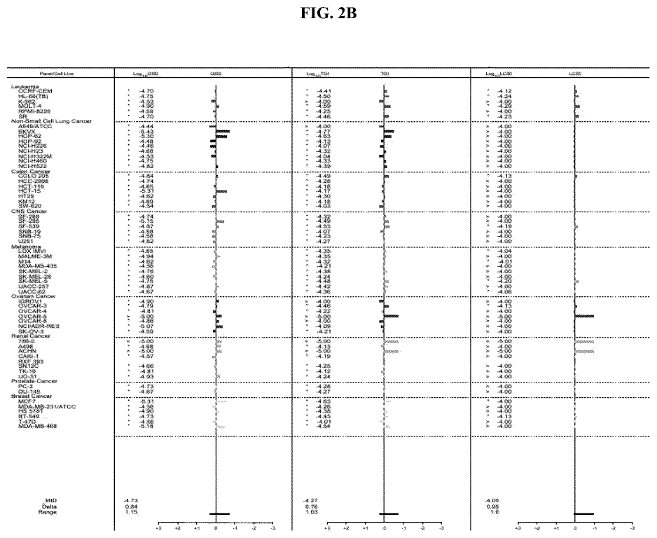

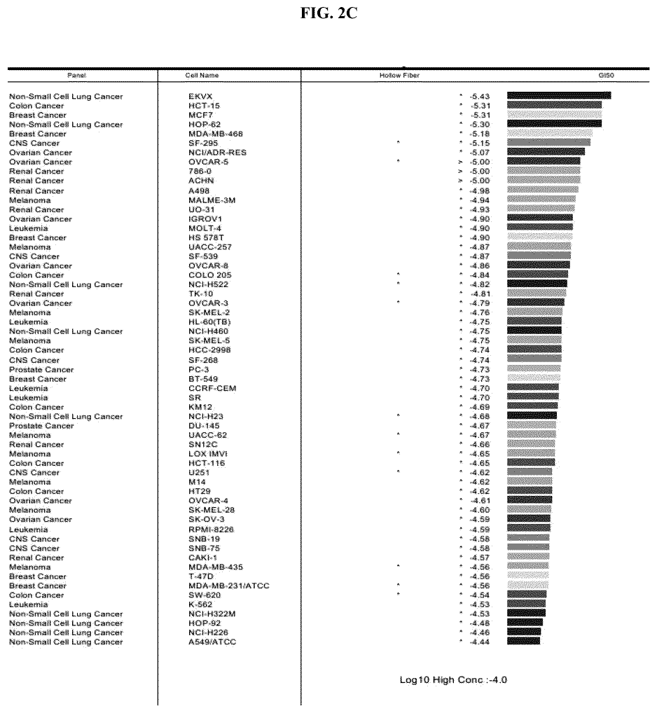

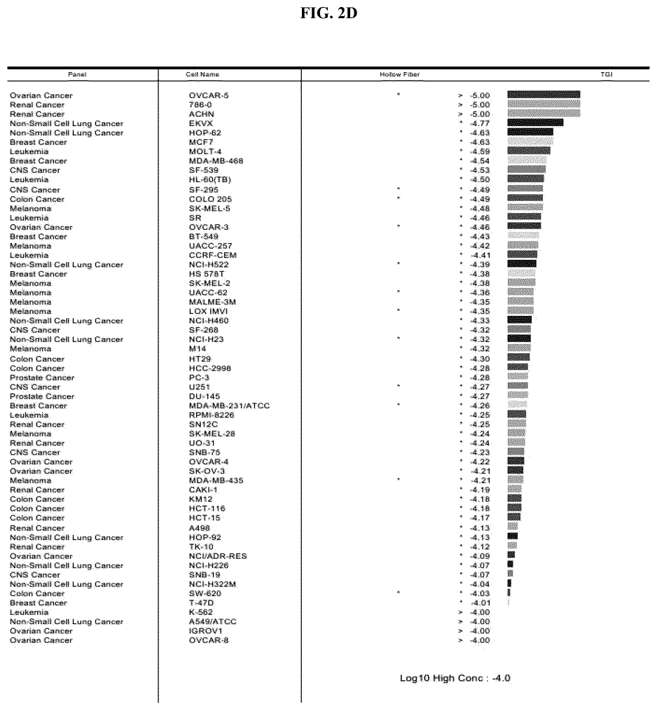

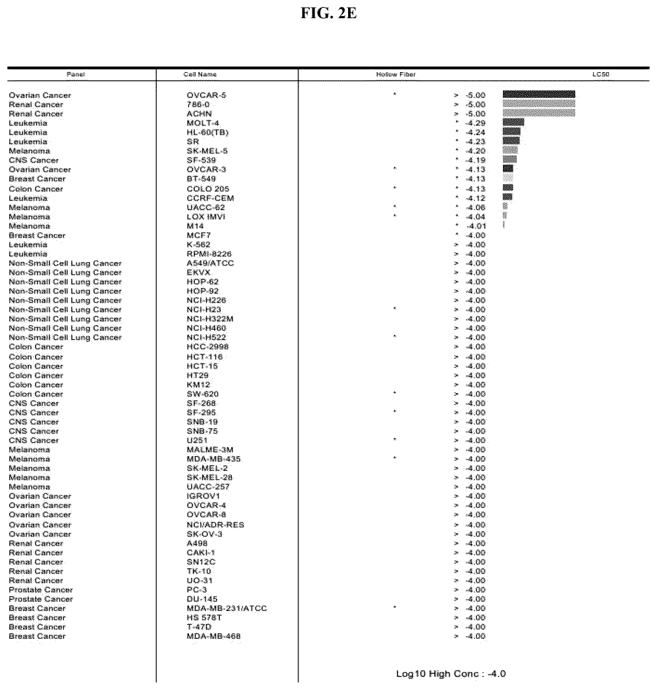

In experiments conducted during development of embodiments herein, AP-7 has proven to be effective against various cancer cell lines (See Table 1 and FIG. 2A-E). Based on the GI50 values, AP-7 is the potent against Non-small Cell Lung Cancer EKVX and HOP-62 cell lines, Colon HCC15 cancer cell line, Brest MCF7 cancer cell line, Central Nervous System SF-2 95 cancer cell line, OVCAR-5, ovarian serous carcinoma, and NCI/Adriamycin resistant ovarian cancer cell lines, and Renal 786-0 and ACHN cancer cell lines. The GI50 values of all 60 cancer cells are in the 3.7-36 micromolar range. Based on the NCI-60 screen and GI50 data, AP-7 inhibits the growth of all 60 cancer cells tested in the Developmental Therapeutics Core at the National Cancer Institute.

TABLE 1
In vitro testing results

| | | | Log t0 Concentration | | | | | | | | | | | | |
|---|---|---|---|---|---|---|---|---|---|---|---|---|---|---|---|
| | Time | | Mean Optical Densities | | | | | Percent Growth | | | | | | | |
| Panel/Cell Line | Zero | Ctrl | −8.0 | −7.0 | −6.0 | −5.0 | −4.0 | −8.0 | −7.0 | −6.0 | −5.0 | −4.0 | GI50 | TGI | LC50 |

Leukemia

| | | | | | | | | | | | | | | | |
|---|---|---|---|---|---|---|---|---|---|---|---|---|---|---|---|
| CCRF-CEM | 0.669 | 3.071 | 3.748 | [symbol] | 3.139 | 3.096 | 0.197 | 128 | 126 | 103 | 101 | −70 | [symbol] | [symbol] 3.88E−5 | [symbol] 7.59E−5 |
| HL-60(TB) | 1.211 | 4.809 | [symbol] | [symbol] | 4.927 | 4.740 | 0.037 | 106 | 113 | 103 | 98 | −97 | [symbol] 1.76E−5 | [symbol] 3.18E−5 | [symbol] 5.75E−5 |
| K-562 | 0.743 | 8.841 | 9.091 | 9.239 | 8.817 | 8.082 | 1.047 | 103 | 105 | 100 | 91 | 4 | [symbol] 2.94E−5 | >1.00E−4 | >1.00E−4 |
| MOLT-4 | 0.968 | 4.328 | 4.736 | 4.856 | 3.830 | 3.224 | 0.023 | 112 | 116 | 85 | 67 | −98 | [symbol] 1.27E−5 | [symbol] 2.56E−5 | [symbol] 5.14E−5 |
| RPMI- [symbol] | 5.319 | 17.501 | 20.719 | 21.605 | 20.390 | [symbol] | 3.338 | 126 | 134 | 124 | 111 | −37 | [symbol] 2.58E−5 | [symbol] | >1.00E−4 |
| 5R | 1.414 | 5.736 | 6.312 | 5.401 | 6.494 | 5.327 | 0.019 | 113 | 115 | 117 | 114 | −99 | [symbol] | [symbol] 3.43E−5 | [symbol] 5.90E−5 |

Non-Small Cell Lung Cancer

| | | | | | | | | | | | | | | | |
|---|---|---|---|---|---|---|---|---|---|---|---|---|---|---|---|
| A549/ATCC | 0.758 | 5.419 | — | 5.172 | 5.500 | 5.932 | 0.897 | — | 95 | 102 | 111 | 3 | [symbol] | >1.00E−4 | >1.00E−4 |
| EKVX | 0.660 | 1.623 | 1.730 | [symbol] | 1.670 | 0.757 | 0.464 | 112 | 104 | 104 | 9 | −30 | [symbol] 3.79E−5 | [symbol] 1.71E−5 | >1.00E−4 |
| HOP- [symbol] | 1.005 | 2.657 | 2.282 | [symbol] | 2.795 | 1.414 | 0.577 | 77 | 100 | 108 | 25 | −43 | [symbol] | [symbol] 2.33E−5 | >1.00E−4 |
| HOP-92 | 8.105 | 13.193 | [symbol] | 14.107 | 14.005 | 14.475 | 8.598 | 112 | 118 | 115 | 125 | −19 | [symbol] | [symbol] 7.43E−5 | >1.00E−4 |
| NCI-H226 | 8.365 | 14.081 | — | 14.725 | 14.728 | 15.165 | 7.651 | — | 111 | 111 | 119 | −9 | [symbol] 3.47E−5 | [symbol] 8.57E−5 | >1.00E−4 |
| NCI-H23 | [symbol] | [symbol] | — | 16.714 | 16.880 | 15.987 | 3.281 | — | 101 | 102 | 94 | −44 | [symbol] 2.05E−5 | [symbol] 4.77E−5 | >1.00E−4 |
| NCI-H322M | 7.676 | 19.182 | — | [symbol] | [symbol] | [symbol] | 7.364 | — | 96 | 96 | 98 | −4 | [symbol] | [symbol] 9.12E−5 | >1.00E−4 |
| NCI-H460 | 1.046 | 15.712 | [symbol] | 15.734 | 16.316 | 12.670 | 0.638 | 102 | 100 | 104 | 79 | −39 | [symbol] 1.77E−5 | [symbol] 4.58E−5 | >1.00E−4 |
| NCI-H522 | 8.799 | 16.586 | [symbol] | 16.889 | 15.524 | 14.349 | 4.472 | 97 | 104 | 80 | 71 | [symbol] | [symbol] 1.52E−5 | [symbol] 4.05E−5 | >1.00E−4 |

Colon Cancer

| | | | | | | | | | | | | | | | |
|---|---|---|---|---|---|---|---|---|---|---|---|---|---|---|---|
| COLO 20 [symbol] | 0.280 | 1.579 | — | 1.456 | 1.205 | 1.217 | 0.090 | — | 90 | 71 | 72 | −58 | [symbol] | [symbol] 3.27E−5 | [symbol] 7.44E−5 |
| HCC-29 [symbol] | 3.611 | 14.809 | — | 12.728 | [symbol] | 12.406 | [symbol] | — | 82 | 80 | 78 | −30 | [symbol] 1.81E−5 | [symbol] 5.25E−5 | >1.00E−4 |
| HCT-11 [symbol] | [symbol] | 5.151 | 4.997 | 6.256 | 5.199 | 4.571 | 0.381 | 97 | 102 | 101 | 88 | −19 | [symbol] 2.25E−5 | [symbol] 5.88E−5 | >1.00E−4 |
| HCT-1 [symbol] | [symbol] | 16.375 | 16.511 | 15.013 | [symbol] | 5.903 | [symbol] | 101 | 90 | 100 | 28 | −6 | [symbol] | [symbol] | >1.00E−4 |
| HT29 | 0.854 | 6.399 | — | [symbol] | [symbol] | 8.895 | 0.460 | — | [symbol] | 94 | 109 | −46 | [symbol] 2.40E−5 | [symbol] 5.04E−5 | >1.00E−4 |
| KM [symbol] 2 | [symbol] | 3.314 | 2.515 | 2.776 | 2.780 | 2.749 | 0.451 | 75 | 81 | 81 | 80 | −17 | [symbol] 2.02E−5 | [symbol] 5.82E−5 | >1.00E−4 |
| SW-620 | 0.670 | [symbol] | 4.680 | 4.737 | 4.652 | 4.304 | 0.854 | 165 | 106 | 104 | 95 | −2 | [symbol] 2.89E−5 | [symbol] 9.44E−5 | >1.00E−4 |

CNS Cancer

| | | | | | | | | | | | | | | | |
|---|---|---|---|---|---|---|---|---|---|---|---|---|---|---|---|
| SF-268 | 1.531 | 4.099 | [symbol] | [symbol] | 3.529 | 3.632 | 0.931 | 88 | 89 | [symbol] | 82 | −39 | [symbol] 1.83E−5 | [symbol] 4.74E−5 | >1.00E−4 |
| SF-295 | 2.119 | [symbol] | 4.967 | 4.974 | 5.027 | 3.231 | 1.304 | 103 | 104 | 105 | 40 | −38 | [symbol] 7.12E−6 | [symbol] 3.25E−5 | >1.00E−4 |
| SF-539 | [symbol] | 14.303 | [symbol] | 13.426 | 13.838 | 11.069 | [symbol] | [symbol] | 92 | [symbol] | 70 | −79 | [symbol] 1.35E−5 | [symbol] 2.95E−5 | [symbol] |
| SNB-19 | 1.305 | 4.995 | 4.577 | [symbol] | 4.941 | 4.651 | 1.222 | 97 | 94 | 98 | 91 | −6 | [symbol] 2.63E−5 | [symbol] 8.59E−5 | >1.00E−4 |
| SNB-75 | 2.147 | 3.701 | 3.625 | 3.727 | 3.772 | 3.858 | 1.458 | 95 | 102 | 104 | 110 | −32 | [symbol] 2.85E−5 | [symbol] 5.94E−5 | >1.00E−4 |
| U251 | 1.315 | 5.843 | — | 5.181 | [symbol] | 6.111 | 0.817 | — | 105 | 103 | 104 | −38 | [symbol] 2.39E−5 | [symbol] 5.40E−5 | >1.00E−4 |

Melanoma

| | | | | | | | | | | | | | | | |
|---|---|---|---|---|---|---|---|---|---|---|---|---|---|---|---|
| LOX IMVI | 0.775 | 6.202 | — | 6.546 | 6.372 | 6.545 | 0.341 | — | 106 | 103 | 106 | −56 | [symbol] 2.22E−5 | [symbol] | [symbol] 9.18E−5 |
| MALME-3M | 9.192 | 14.357 | 14.865 | 15.458 | 15.768 | 12.018 | 6.425 | 110 | 121 | 128 | 55 | −30 | [symbol] 1.14E−5 | [symbol] 4.42E−5 | >1.00E−4 |
| M14 | [symbol] | 15.354 | 15.781 | 15.822 | [symbol] | [symbol] | 2.430 | 104 | 104 | 108 | 112 | −52 | [symbol] 2.40E−5 | [symbol] 4.63E−5 | [symbol] 9.73E−5 |
| MDA-MB-435 | [symbol] | 3.735 | 4.170 | 4.139 | 3.997 | 4.005 | 0.929 | [symbol] | 117 | 111 | 111 | [symbol] | [symbol] 2.73E−5 | [symbol] 6.22E−5 | >1.00E−4 |
| SK-MEL-2 | 2.498 | 5.955 | — | 5.762 | 5.794 | 5.270 | 1.292 | — | 95 | [symbol] | 80 | −48 | [symbol] 1.72E−5 | [symbol] 4.22E−5 | >1.00E−4 |
| SK-MEL- [symbol] | 2.074 | 4.570 | 4.178 | [symbol] | 4.474 | 4.733 | 1.371 | 84 | 99 | 96 | 107 | −34 | [symbol] 2.53E−5 | [symbol] | >1.00E−4 |
| SK-MEL-5 | 4.050 | 17.981 | — | 17.497 | 15.403 | 17.479 | 0.503 | — | 97 | 82 | 96 | −88 | [symbol] 1.78E−5 | [symbol] 3.33E−5 | [symbol] 6.24E−5 |
| UACC-257 | 2.506 | 5.926 | 5.898 | 6.504 | 6.187 | 4.730 | 1.305 | 99 | 117 | 108 | 65 | −48 | [symbol] | [symbol] 3.77E−5 | >1.00E−4 |
| UACC-62 | 0.762 | 2.454 | 2.775 | 2.932 | 2.705 | 2.532 | 0.311 | 119 | [symbol] | 116 | 105 | −59 | [symbol] 2.15E−5 | [symbol] 4.35E−5 | [symbol] 8.79E−5 |

Ovarian Cancer

| | | | | | | | | | | | | | | | |
|---|---|---|---|---|---|---|---|---|---|---|---|---|---|---|---|
| IGROV1 | 1.580 | 4.905 | 5.201 | [symbol] | 5.416 | 3.422 | 1.614 | 109 | 111 | 115 | 55 | 1 | [symbol] 1.25E−5 | >1.00E−4 | >1.00E−4 |
| OVCAR-3 | 3.561 | 10.627 | 11.407 | [symbol] | 9.942 | 9.315 | 1.052 | 111 | 102 | 90 | 52 | −70 | [symbol] 1.62E−5 | [symbol] 3.45E−5 | [symbol] 7.34E−5 |
| OVCAR-4 | 6.720 | 10.583 | 11.497 | 11.987 | 11.448 | 10.578 | 4.854 | 115 | 127 | 114 | 100 | −28 | [symbol] 2.47E−5 | [symbol] | >1.00E−4 |
| OVCAR-5 | 7.651 | 18.015 | 19.314 | 19.391 | 18.965 | [symbol] | — | 113 | 113 | 109 | [symbol] | — | >1.00E−5 | >1.00E−5 | >1.00E−5 |
| OVCAR-8 | 1.001 | 4.137 | 4.484 | [symbol] | 4.407 | 2.809 | 1.071 | 111 | 107 | 109 | 58 | 2 | [symbol] 1.37E−5 | >1.00E−4 | >1.00E−4 |
| NCI/ADR-RES | 8.594 | 17.107 | 17.930 | 18.391 | [symbol] | 11.378 | [symbol] | 108 | 112 | 115 | [symbol] | −4 | [symbol] | [symbol] 8.14E−5 | >1.00E−4 |
| SK-OV-3 | 3.365 | 8.953 | — | 8.406 | 8.890 | 9.144 | 2.450 | — | 90 | 99 | 104 | −28 | [symbol] 2.58E−5 | [symbol] 6.18E−5 | >1.00E−4 |

Renal Cancer

| | | | | | | | | | | | | | | | |
|---|---|---|---|---|---|---|---|---|---|---|---|---|---|---|---|
| 786-0 | 1.052 | 5.162 | 5.491 | 5.493 | 5.390 | [symbol] | — | 108 | 108 | 106 | 64 | — | >1.00E−5 | >1.00E−5 | >1.00E−5 |
| A498 | 4.985 | 13.468 | 13.836 | [symbol] | 14.796 | 9.352 | 4.596 | 104 | 106 | 116 | 52 | [symbol] | [symbol] 1.08E−5 | [symbol] | >1.00E−4 |
| ACHN | 1.200 | 4.747 | 4.954 | 5.087 | 5.105 | [symbol] | — | 106 | 109 | 110 | [symbol] | — | >1.00E−5 | >1.00E−5 | >1.00E−5 |
| CAKI-1 | 1.194 | 5.150 | 5.534 | 5.508 | 5.437 | 5.385 | 0.903 | 110 | 109 | 107 | 106 | −24 | [symbol] 2.69E−5 | [symbol] | >1.00E−4 |
| RXF 393 | 2.354 | — | — | — | — | — | — | — | — | — | — | — | — | — | — |
| SN12C | 1.357 | 4.472 | 4.530 | 4.308 | 4.297 | 4.208 | 0.947 | 102 | 95 | 94 | 92 | −30 | [symbol] | [symbol] | >1.00E−4 |
| TK-10 | 7.325 | 15.429 | 16.520 | [symbol] | [symbol] | 12.494 | [symbol] | 113 | 116 | 109 | 54 | [symbol] | [symbol] 1.55E−5 | [symbol] | >1.00E−4 |
| UO-31 | [symbol] | 4.974 | 5.079 | 5.405 | 5.447 | 3.455 | 1.293 | 103 | 113 | 114 | 55 | −17 | [symbol] 1.19E−5 | [symbol] 5.76E−5 | >1.00E−4 |

Prostate Cancer

| | | | | | | | | | | | | | | | |
|---|---|---|---|---|---|---|---|---|---|---|---|---|---|---|---|
| PC-3 | 4.573 | 18.261 | 17.953 | 19.719 | 19.498 | 15.410 | 3.195 | 98 | 111 | 109 | 79 | −30 | [symbol] 1.85E−5 | [symbol] 5.30E−5 | >1.00E−4 |
| DU-145 | 1.298 | 4.191 | 4.358 | 4.452 | [symbol] | 3.922 | [symbol] | 106 | 109 | 105 | 91 | −33 | [symbol] 2.13E−5 | [symbol] 5.41E−5 | >1.00E−4 |

Breast Cancer

| | | | | | | | | | | | | | | | |
|---|---|---|---|---|---|---|---|---|---|---|---|---|---|---|---|
| MCF7 | 2.355 | [symbol] | 14.205 | 13.950 | 13.319 | 5.785 | 1.176 | 103 | 101 | 95 | 30 | −50 | [symbol] | [symbol] | [symbol] 9.99E−5 |
| MDA-MB- | 5.359 | 10.500 | 12.105 | 12.070 | [symbol] | 12.211 | 2.962 | 124 | 123 | 120 | 126 | −45 | [symbol] 2.78E−5 | [symbol] 5.48E−5 | >1.00E−4 |
| 231/ATCC | | | | | | | | | | | | | | | |
| HS 578T | 1.822 | 3.949 | — | 3.305 | 3.701 | 3.109 | 1.182 | — | 70 | 88 | 50 | −38 | [symbol] 1.27E−5 | [symbol] 4.21E−5 | >1.00E−4 |
| BT-549 | [symbol] | 14.245 | 13.878 | [symbol] | 14.344 | 13.797 | 1.852 | 95 | 94 | 101 | 94 | −72 | [symbol] 1.85E−5 | [symbol] 3.69E−5 | [symbol] |
| T-47D | 4.991 | 12.528 | — | 12.492 | 12.625 | 11.704 | 4.963 | — | 99 | 101 | 89 | −1 | [symbol] 2.73E−5 | [symbol] 9.58E−5 | >1.00E−4 |
| MDA-MB- [symbol] | 7.624 | 8.936 | — | 8.695 | 8.823 | 8.163 | 3.979 | — | 80 | 90 | 41 | −48 | [symbol] | [symbol] 2.90E−5 | >1.00E−4 |

[symbol]

indicates data missing or illegible when filed

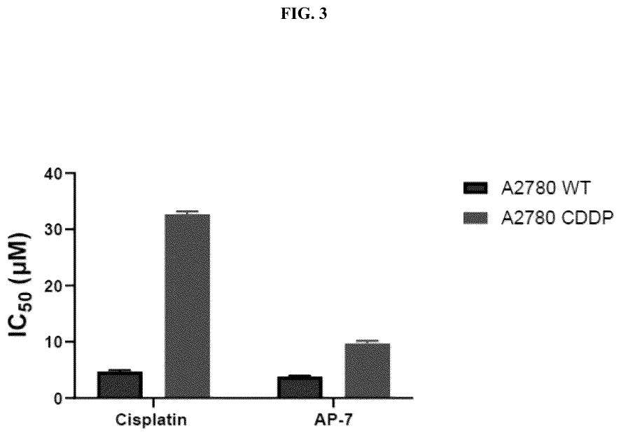

IC₅₀ values were measured for AP-7 in A2780 WT and A2780 CDDP ovarian cancer cell lines (FIG. 3).

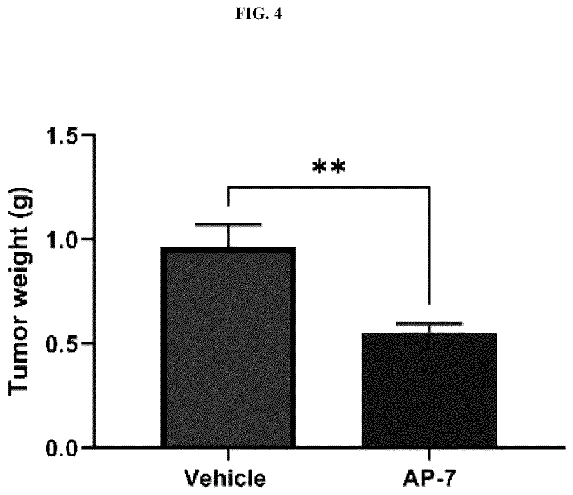

Based on the TGI values, Arsenoplatin-7 was most potent against OVCAR-5, a very aggressive and invasive ovarian cancer cell line. Experiments were conducted during development of embodiments herein to test Arsenoplatin-7 in the patient derived xenograft (PDX) model of ovarian cancer, where it decreased the tumor size statistically significantly compared to the control (FIG. 4).

The maximum tolerated dose studies show that AP-7 is significantly less toxic than the FDA-approved Cisplatin. The MTD for AP-7 is 90 mg/kg compared to Cisplatin (4-6 mg/kg); (FIG. 5).

### Example 4

### Synthesis of Arsenoplatin-9 (AP-9)

Arsenoplatin-9 (AP-9) was synthesized in the reaction between Arsenoplatin-1 (AP-1) and 2-methylimidazole in a 1:1 mole ratio in a methanol-water mixture using the same solvent system and pH range as described in Example 1 for Arsenoplatin-7. The general procedure, including the use of a methanol-water mixture as the solvent system, pH adjustment, and slow evaporation for crystal growth, was the same as that described in Example 1.

### Example 5

### Single Crystal X-Ray Structure Determination of AP-9

A single crystal x-ray structure was obtained for AP-9. The crystal structure of AP-9 (FIG. 8) confirms the structural assignment of AP-9 as a 2-methylimidazole derivative of Arsenoplatin-1, with the 2-methylimidazole ligand coordinated to the platinum (II) center through a ring nitrogen atom. The crystal structure reveals a structural pattern consistent with that observed for AP-7 (Example 2), with the platinum (II) center coordinated to the nitrogenous base, bridged to the arsenic center by two acetoamidate ligands. Examination of the As—O bond lengths in the crystal structure indicates that one As—O bond is consistent with a single bond (As—OH) and the other is consistent with a double bond (As═O), supporting the existence of a tautomeric form in which one arsenic-bound oxygen is deprotonated.

### Example 6

### Synthesis of Arsenoplatin-11 (AP-11)

Arsenoplatin-11 (AP-11) was synthesized in the reaction between Arsenoplatin-1 (AP-1) and 2-ethylimidazole in a 1:1 mole ratio in a methanol-water mixture using the same solvent system and pH range as described in Example 1 for Arsenoplatin-7. The general procedure, including the use of a methanol-water mixture as the solvent system, pH adjustment, and slow evaporation for crystal growth, was the same as that described in Example 1.

### Example 7

### Single Crystal X-Ray Structure Determination of AP-11

A single crystal x-ray structure was obtained for AP-11. The crystal structure of AP-11 (FIG. 9) confirms the structural assignment of AP-11 as a 2-ethylimidazole derivative of Arsenoplatin-1, with the 2-ethylimidazole ligand coordinated to the platinum (II) center through a ring nitrogen atom. The crystal structure is fully consistent with the structural pattern observed for AP-7 (Example 2) and AP-9 (Example 5). The fully labeled crystal structure shows the expected connectivity: Pt1 is coordinated to N3 and N4 of the two bridging acetoamidate ligands and to N2 of the 2-ethylimidazole ring, while As1 is bridged to Pt1 through the amidate oxygen atoms 01 and 02. The arsenic center bears additional oxygen atoms 03 and 04, with bond length differences consistent with one As—OH bond and one As═O bond, supporting the tautomeric assignment described herein. A co-crystallized water molecule (05) is also present in the crystal lattice.

### Example 8

### Structural Comparison of AP-7, AP-9, and AP-11

Comparison of the single crystal x-ray structures of AP-7 (Example 2), AP-9 (Example 5), and AP-11 (Example 7) demonstrates that all three derivatives share the same structural pattern. In each compound, the platinum (II) center is coordinated to the nitrogenous base ligand and bridged to the arsenic center by two acetoamidate ligands. In each structure, examination of the As—O bond lengths indicates that one arsenic-oxygen bond is consistent with a single bond (As—OH) and the other is consistent with a double bond (As═O). The structural conservation across the three derivatives, which differ only in the substituent at the 2-position of the imidazole ring (H for AP-7, methyl for AP-9, ethyl for AP-11), supports the general applicability of the synthetic method described in Example 1 to 2-substituted imidazole derivatives and confirms the tautomeric forms described in the detailed description.

### Example 9

### Synthesis of Nitrogenous Base Derivatives of Arsenoplatin-1 (General Method)

Additional nitrogenous base derivatives of Arsenoplatin-1 can be prepared by the general method described in Examples 1, 4, and 6. A nitrogenous base A selected from Table 2 is reacted with Arsenoplatin-1 (AP-1) in a 1:1 mole ratio in a methanol-water mixture (v/v 1:1). The pH is adjusted to about 7.0 to about 8.0, and the reaction mixture is maintained at about 40° C. to about 60° C. with reflux. After the reaction, the solution is filtered, transferred to a suitable vessel, and subjected to slow evaporation to yield crystals of the corresponding AP-A compound.

TABLE 2
Exemplary nitrogenous bases for synthesis of AP-A derivatives

| Nitrogenous Base A | Expected Product |
|---|---|
| Imidazole | AP-7 |
| 2-Methylimidazole | AP-9 |
| 2-Ethylimidazole | AP-11 |
| 4-Methylimidazole | AP-A (4-MeIm) |
| 5-Methylimidazole | AP-A (5-MeIm) |
| 1-Methylimidazole | AP-A (1-MeIm) |
| 1,2-Dimethylimidazole | AP-A (1,2-DMIm) |
| Pyrazole | AP-A (Pyr) |
| 1,2,4-Triazole | AP-A (Tri) |

Without being bound by theory, based on the successful synthesis of AP-7, AP-9, and AP-11 under the same general conditions, it is expected that other small, unsubstituted or alkyl-substituted five-membered nitrogen-containing heterocycles will react with AP-1 under similar conditions to form the corresponding AP-A derivatives.

1. A composition comprising a compound of the structure: 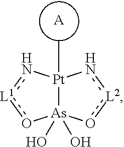
or a tautomer thereof;
wherein A is a nitrogenous base and L¹ and L² are bridging amidate ligands.

2. The composition of claim 1, wherein the tautomer is of the structure:

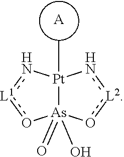

3. The composition of claim 1, wherein A is a five-membered nitrogen-containing heterocycle.

4. The composition of claim 3, wherein A is imidazole or an imidazole derivative.

5. The composition of claim 4, wherein A is selected from 2-methylimidazole, 4-methylimidazole, 5-methylimidazole, 2-ethylimidazole, 1-methylimidazole, 1,2-dimethylimidazole.

6. The composition of claim 1, wherein A is selected from imidazole, 2-methylimidazole, 4-methylimidazole, 5-methylimidazole, 2-ethylimidazole, 1-methylimidazole, 1,2-dimethylimidazole, histidine, histamine, clotrimazole, ketoconazole, metronidazole, miconazole, pyrazole, 1,2,3-triazole, 1,2,4-triazole, fluconazole, itraconazole, tetrazole, thiazole, oxazole, benzimidazole, albendazole, mebendazole, benzotriazole, 1,2,4-oxadiazole, adenine, guanine, cytosine, thymine, uracil, pyrrole, pyridine, imidazopyridine, and zolpidem.

7. The composition of claim 1, wherein L¹ and L² are C(O), C—R¹ or C—R², and wherein R¹ and R² are independently selected from hydrogen, halogen, cyano, keto, ester, ether, thiol, thioether, thioester, imino, C₁-C₁₀ alkyl, C₁-C₁₀ alkenyl, alkynyl, alkoxyl, amino, amidyl, immino, sulfonyl, sulfoxyl, phosphoryl, phosphoryl ester, glycosyl, aryl, C₃-C₁₅ cycloalkyl, heteroaryl, and C₃-C₁₅ heterocycloalkyl.

8. The composition of claim 7, wherein R¹ and R² are each independently selected from a C₁-C₁₀ alkyl.

9. The composition of claim 1, wherein L¹ and L² are identical functional groups.

10. The composition of claim 9, wherein L¹ and L² are both —CH3, and the compound is of the structure: 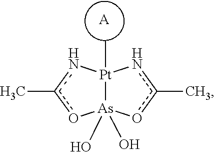
or a tautomer thereof.

11. The composition of claim 10, wherein the tautomer is of the structure:

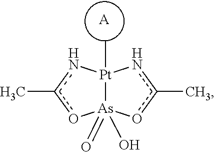

12. The composition of claim 10, wherein A is imidazole and the compound is of the structure: 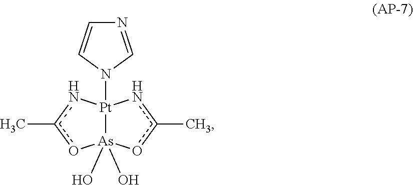
or a tautomer thereof.

13. The composition of claim 12, wherein the tautomer of AP-7 is of the structure:

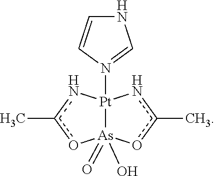

14. The composition of claim 10, wherein A is 2-methylimidazole and the compound is of the structure: 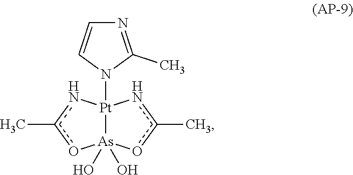
or a tautomer thereof.

15. The composition of claim 14, wherein the tautomer of AP-9 is of the structure:

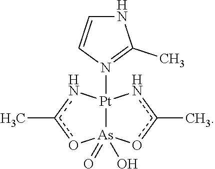

16. The composition of claim 10, wherein A is 2-ethylimidazole and the compound is of the structure: 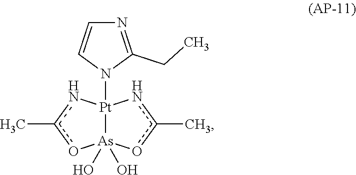
or a tautomer thereof.

17. The composition of claim 16, wherein the tautomer of AP-11 is of the structure:

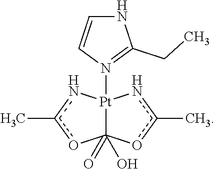

18. A pharmaceutical composition comprising the composition of claim 1, and a pharmaceutically acceptable carrier.

19-21. (canceled)

22. A method of treating cancer in a subject in need thereof, comprising administering to the subject a therapeutically effective amount of the pharmaceutical composition according to claim 18.

23-30. (canceled)

31. A method of making a composition according to claim 1, comprising reacting Arsenoplatin-1 (AP-1) with a nitrogenous base A in a solvent system to form the compound.

32-34. (canceled)
